(19) United States
(12) Patent Application Publication
(10) Pub. No.: US 2014/0031643 A1
(43) Pub. Date: Jan. 30, 2014

(54) HEART FAILURE PATIENTS STRATIFICATION

(71) Applicant: Cardiac Pacemakers, Inc., St. Paul, MN (US)

(72) Inventors: Qi An, Blaine, MN (US); Yi Zhang, Plymonth, MN (US); Viktoria A. Averina, Roseville, MN (US); Pramodsingh Hirasingh Thakur, White Bear Lake, MN (US); Robert J. Sweeney, Woodbury, MN (US)

(21) Appl. No.: 13/912,541

(22) Filed: Jun. 7, 2013

Related U.S. Application Data
(60) Provisional application No. 61/676,679, filed on Jul. 27, 2012.
(60) Provisional application No. 61/768,821, filed on Feb. 25, 2013.

(51) Int. Cl. A61B 5/00; A61B 5/0452; A61B 5/145; A61B 7/00; A61B 5/0205

(52) U.S. Cl. CPC A61B 5/7275; A61B 7/00; A61B 5/0205; A61B 5/14546; A61B 5/746; A61B 5/0452
USPC 600/309; 600/508; 600/528; 600/484

(57) ABSTRACT

A system, apparatus and method are provided to quantify a risk of worsening heart failure for subject using at least one physiological sensor circuit such as, for example, a heart sound sensor, a respiration sensor, a cardiac activity sensor, or other sensor circuit. A central tendency measurement of the at least one physiological sensor can be used to quantify the risk of worsening heart failure of the subject.

### CLAIM OF PRIORITY

This application claims the benefit of U.S. Provisional Patent Application Ser. No. 61/676,679, filed on Jul. 27, 2012 and also claims the benefit of U.S. Provisional Patent Application Ser. No. 61/768,821, filed on Feb. 25, 2013, the benefit of priority of each of which is claimed hereby, and each of which are incorporated by reference herein in its entirety.

### BACKGROUND

Ambulatory medical devices include implantable medical devices (IMDs) and wearable medical devices. Some examples of IMDs include cardiac function management (CFM) devices such as implantable pacemakers, implantable cardioverter defibrillators (ICDs), cardiac resynchronization therapy devices (CRTs), and devices that include a combination of such capabilities. IMDs can be used to treat patients or subjects using electrical or other therapy or to aid a physician or caregiver in patient diagnosis through internal monitoring of the condition of a patient or subject. The devices may include one or more electrodes in communication with one or more sense amplifiers to monitor electrical heart activity within a patient, and often include one or more sensors to monitor one or more other internal patient parameters. Other examples of IMDs include implantable diagnostic devices, implantable drug delivery systems, or implantable devices with neural stimulation capability.

Wearable medical devices include wearable cardioverter defibrillators (WCDs) and wearable diagnostic devices (e.g., an ambulatory monitoring vest). WCDs can be monitoring devices that include surface electrodes. The surface electrodes are arranged to provide one or both of monitoring to provide surface electrocardiograms (ECGs) and delivering cardioverter and defibrillator shock therapy. Ambulatory medical devices can also include one or more sensors to monitor one or more physiologic parameters of a subject.

Some ambulatory medical devices include one or more sensors to monitor different physiologic aspects of the patient. The devices may derive measurements of hemodynamic parameters related to chamber filling and contractions or other physiological parameters from electrical signals provided by such sensors. Sometimes patients who are prescribed these devices have experienced repeated heart failure (HF) decompensation or other events associated with worsening HF (WHF). Symptoms associated with WHF may include pulmonary and/or peripheral edema, dilated cardiomyapathy, or ventricular dilation. Some patients with chronic HF may experience an acute HF event. Device-based monitoring can identify those HF patients having a risk of experiencing an acute HF event.

### Overview

This document relates generally to systems, devices, and methods for detection of heart failure. An apparatus example includes at least a first physiological sensor circuit configured to generate a first physiological signal that is representative of cardiovascular function of a subject, and a control circuit communicatively coupled to the first physiological sensor circuit. The control circuit can include a signal processing circuit and a risk circuit. The signal processing circuit may be configured to determine a first physiological measurement using the first physiological sensor signal and determine a plurality of the first physiological measurements using a plurality of first physiological signals produced over a first specified time period, and determine a central tendency measurement of the plurality of physiological measurements. The risk circuit may be configured to quantify a risk of WHF for the subject using the determined central tendency measurement, such as for example by including comparing the determined central tendency measurement to one or more criteria indicative of risk of WHF. The control circuit may be configured to generate an indication of risk of WHF according to a comparison of the determined central tendency measurement to the one or more criteria indicative of risk of WHF.

This section is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, the various examples discussed in the present document.

FIG. 1 is an illustration of portions of a system that includes an ambulatory medical device.

FIG. 2 is an illustration of portions of another system that includes an ambulatory medical device.

FIG. 3 is a flow diagram of a method of operating an ambulatory medical device to monitor a subject for risk of WHF.

FIG. 4 is an example of graphs related to the likelihood of an HF patient not experiencing WHF.

FIG. 5 shows an example of a graph of related to a regression model of S3 energy data for a patient population.

FIG. 6 shows an example of assessing the risk of WHF using energy of the S3 heart sound.

FIG. 7 shows an example of portions of an ambulatory medical device that assesses the risk of WHF for a subject.

FIG. 8 shows an example of assessing the risk of WHF using S3 energy and respiratory rate variation.

FIG. 9 shows an example of assessing the risk of WHF using S3 energy and history of HF admissions.

### DETAILED DESCRIPTION

An ambulatory medical device is capable of moving about with the subject, such as chronically during activities of daily living. Such a device may include one or more of the features, structures, methods, or combinations thereof described herein. For example, a cardiac monitor or a cardiac stimulator may be implemented to include one or more of the advantageous features or processes described below. It is intended that such a monitor, stimulator, or other implantable or partially implantable device need not include all of the features described herein, but may be implemented to include selected features that provide for unique structures or functionality. Such a device may be implemented to provide a variety of therapeutic or diagnostic functions.

Systems and methods are described herein for improved assessment of WHF of a patient. Patients with chronic HF may experience an acute HF event (e.g., a HF decompensation event). Due to limited health care resources, it may be desirable to identify those patients who are at risk and allocate medical care resources accordingly. A device-generated risk index for HF may help identify those patients with a relatively high risk of WHF, or alternatively identify those patients with a relatively low risk of WHF, and allocate resources for monitoring and treating HF while maintaining similar quality of health care to all HF patients.

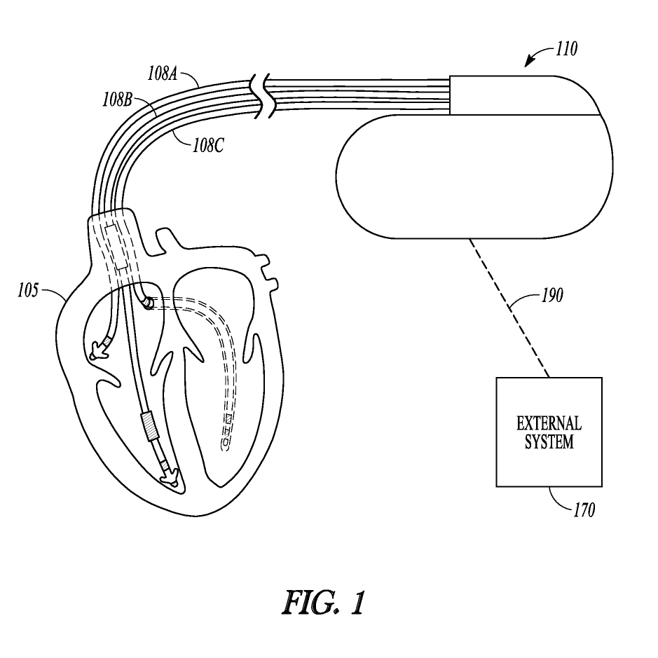

Medical electronic systems can be used to obtain information related to a patient's physiologic condition. FIG. 1 is an illustration of portions of a system that includes an IMD 110. Examples of IMD 110 can include, without limitation, a pacemaker, a defibrillator, a cardiac resynchronization therapy (CRT) device, or a combination of such devices. The IMD 110 can be coupled by one or more leads 108A-C to heart 105. Cardiac leads 108A-C include a proximal end that is coupled to IMD 110 and a distal end, coupled by electrical contacts or “electrodes” to one or more portions of a heart 105. The electrodes can be configured to deliver an electrical stimulus to the heart 105 to provide cardioversion, defibrillation, pacing, or resynchronization therapy, or combinations thereof. The electrodes may be electrically coupled to sense amplifiers to sense electrical cardiac signals.

Medical electronic systems can also include other physiologic sensors to monitor other physiologic parameters. For example, a wearable device can include surface electrodes (e.g., electrodes for skin contact) to sense a cardiac signal such as an electrocardiograph (ECG). In another example, a physiologic sensor can include a heart sound sensor circuit that senses heart sounds. Heart sounds are associated with mechanical vibrations from activity of a subject's heart and the flow of blood through the heart. Heart sounds recur with each cardiac cycle and can be separated and classified according to the activity associated with the vibration. The first heart sound (S1) is the vibrational sound made by the heart during tensing of the mitral valve. The second heart sound (S2) marks the closing of the aortic valve and the beginning of diastole. The third heart sound (S3) and fourth heart sound (S4) are related to filling pressures of the left ventricle during diastole. A heart sound sensor circuit can produce an electrical physiologic signal which is representative of mechanical activity of a patient's heart. A heart sound sensor circuit can be disposed in the heart, near the heart, in an IMD, in a wearable patch on a patient's skin, or in another location where the acoustic energy of heart sounds can be sensed. In some examples, the heart sound sensor circuit includes an accelerometer disposed in the IMD of FIG. 1. In another example, the heart sound sensor circuit includes a microphone to sense acoustic energy or vibrations of the heart 105.

As shown in FIG. 1, the system may include a medical device programmer or other external system 170 that communicates with the IMD 110 via wireless signals 190. In some examples, the wireless communications can include using radio frequency (RF). However, other suitable telemetry signals can be used. The physiological sensors can be included in a diagnostic-only device. The diagnostic-only device may be subcutaneously implantable with a one or more leads that can be transvenous leads or non-transvenous leads. The physiological sensors may be included in a wearable surface ICD (S-ICD) that includes patch electrodes that contact the skin of the patient. In still another example, the physiological sensors can be included in a neural stimulator device that provides an electrical stimulus to nerve sites such as a vagal nerve or the carotid sinus for example.

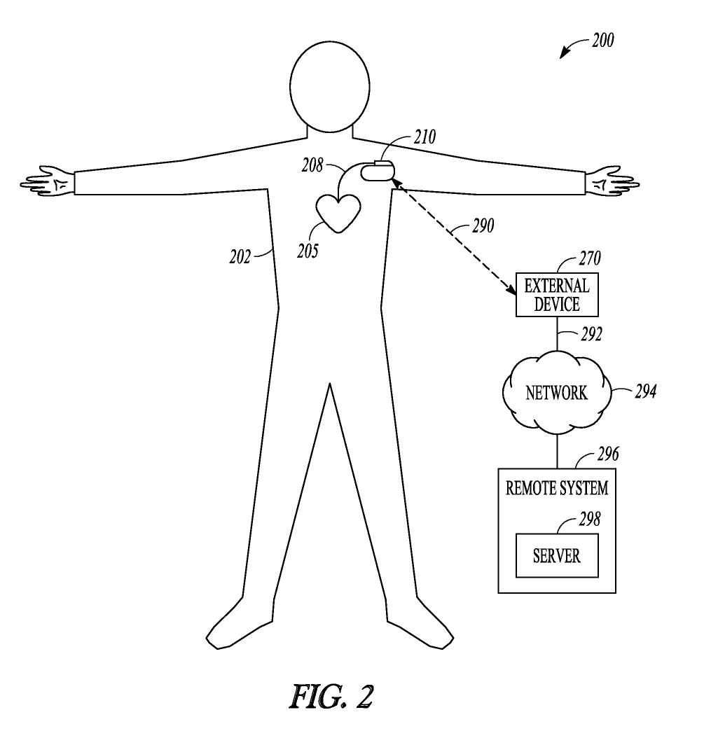

FIG. 2 is an illustration of portions of a system 200 that uses an IMD, wearable medical device, or other ambulatory medical device 210 to provide a therapy to a patient 202. The system 200 may include an external device 270 that communicates with a remote system 296 via a network 294. The network 294 can be a communication network such as a phone network or a computer network (e.g., the internet). In some examples, the external device 270 includes a repeater and communicates via the network using a link 292 that can be wired or wireless. In some examples, the remote system 296 provides patient management functions and can include one or more servers 298 to perform the functions. Device communications can allow for remote monitoring for the risk of an acute HF event. Device-based sensor data may provide a continuous indicator of a subject's HF status; in contrast to traditional clinical diagnostics that provide only a snapshot of the status when the subject is examined in a clinical setting.

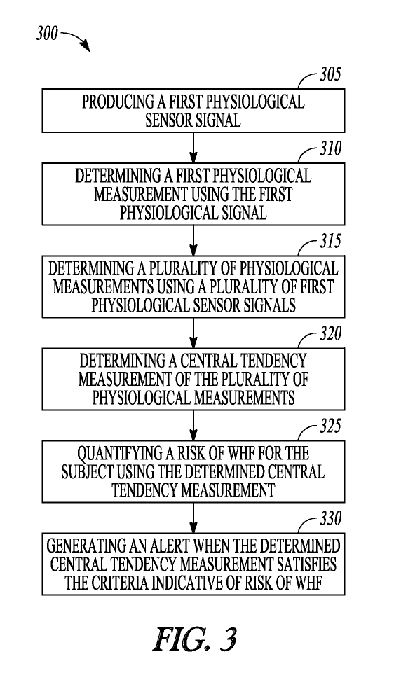

FIG. 3 is a flow diagram of a method 300 of operating an ambulatory medical device to monitor a subject for risk of WHF. The method 300 can include collecting data from one or more sensors such as device-based sensors. The sensors sense physiological properties of the patient. Some examples of the sensors include a heart sound sensor, a respiration sensor, a posture sensor, an intra-thoracic impedance sensor, a cardiac signal sensor, and a chemical sensor. The sensors may be included in one or more of an IMD (e.g., a pacemaker, ICD, S-ICD, diagnostic-only device, neurostimulator, etc.) or may be provided as a wearable device or patch.

The method 300 may quantify the risk of acute HF events within a specified time frame (e.g., over the next month, three months, six months, or twelve months) for the subject. In some circumstances, the risk of acute HF events may be quantified using the collected data from one or more sensors, historical HF information for the subject, or both the collected data and the historical information.

At block 305, a physiological sensor signal can be generated by the ambulatory medical device that is based, at least in part, on a physiological parameter sensed by a physiological sensor. The physiological sensor signal can be representative of cardiovascular function of a subject. A non-exhaustive list of examples of a physiological sensor signal includes a heart sound signal, a respiration signal, a cardiac activity signal, and a biomarker signal. As explained previously herein, a heart sound signal can be representative of mechanical activity of a heart of the subject and a respiration signal can be representative of respiration of the subject. A cardiac activity signal can be representative of electrical cardiac activity of the subject and can include one or more fiducial features corresponding to cardiac activation, such as a QRS complex for example that is associated with activation of the ventricles. A biomarker signal is representative of a level of biomarker in the subject. The biomarker can include B-type Naturetic Peptide (BNP). BNP is secreted by a ventricle of the heart in response to excessive stretching of the myocardium due to HF. In certain examples, the biomarker includes an N-terminal amino acid secreted with BNP (NT-Pro-BNP). In some examples, the method at block 305 can include producing a combination of any of the physiological sensor signals described herein.

At block 310, a first physiological measurement is determined using the physiological sensor signal. In some examples, a central tendency of the physiological sensor signal can be determined and the physiological parameter is measured from the central tendency signal, but this is not required. A non-exhaustive list of examples of the physiological measurement include a measure of post-S2 heart sound energy (e.g., S3 heart sound energy), a measure of respiration rate, a measure of a level of biomarker, a measure of a time interval between fiducial features in one or more physiological sensor signals, or a ratio of such measured time intervals.

According to some examples, the physiological sensor signal used to determine the parameter is generated from multiple signals sensed by the physiological sensor. For instance, the physiological sensor signal may generate a first type of physiological sensor signal. A central tendency signal can be produced (e.g., by ensemble averaging) from a plurality of signals of this type that were obtained for a number of cardiac cycles (e.g., 8 to 16 cardiac cycles) or interval of time (e.g., 30 seconds). Using a central tendency signal may be more helpful for prediction of WHF in contrast to one instantaneous signal. A single instantaneous signal may include factors that overly influence the analysis. The physiological measurement can be determined using a physiological sensor signal that is a central tendency sensor signal.

At block 315, a plurality of physiological sensor signals can be produced over a specified (e.g., programmed) first time period and a plurality of physiological measurements can be determined using the plurality of physiological sensor signals. In some examples, the first time period is a number of days (e.g., 1 day, 5 days, a week, 10 days, a month, etc.). The plurality of signals may be of different types of physiological signals.

At block 320, a central tendency of the plurality of physiological measurements can be determined to generate a central tendency measurement. Some examples of a central tendency measurement include an average of the physiological measurements obtained for the specified time period or a median value of the physiological measurements. Note that the time period for determining a central tendency measurement (e.g., a day or more) has a greater time scale than the time period used to produce a central tendency signal (e.g., 30 seconds). The time periods can be specified by programming, but this is not required.

At block 325, a risk of WHF for the subject is quantified using the determined central tendency measurement. Quantifying the risk can include comparing the determined central tendency measurement to one or more criteria indicative of risk of WHF. For instance, the determined central tendency measurement may be an average of measurements of post-S2 heart sound magnitude taken over a 10-day period. If the averaged measurement exceeds a WHF detection threshold magnitude value, the subject may be assigned a higher risk score or assigned a high risk category. In this way, the risk of experiencing WHF can be stratified.

Determining the central tendency of a physiologic measurement can be useful in measurements used to stratify risk of WHF according to the physiological data. This is because the physiological measurements may include temporary variations in the measurements due to heart rate change, due to a change in a signal generated by the physiological sensor, or due to a change in a measurement over the course of a day, may confound the stratification.

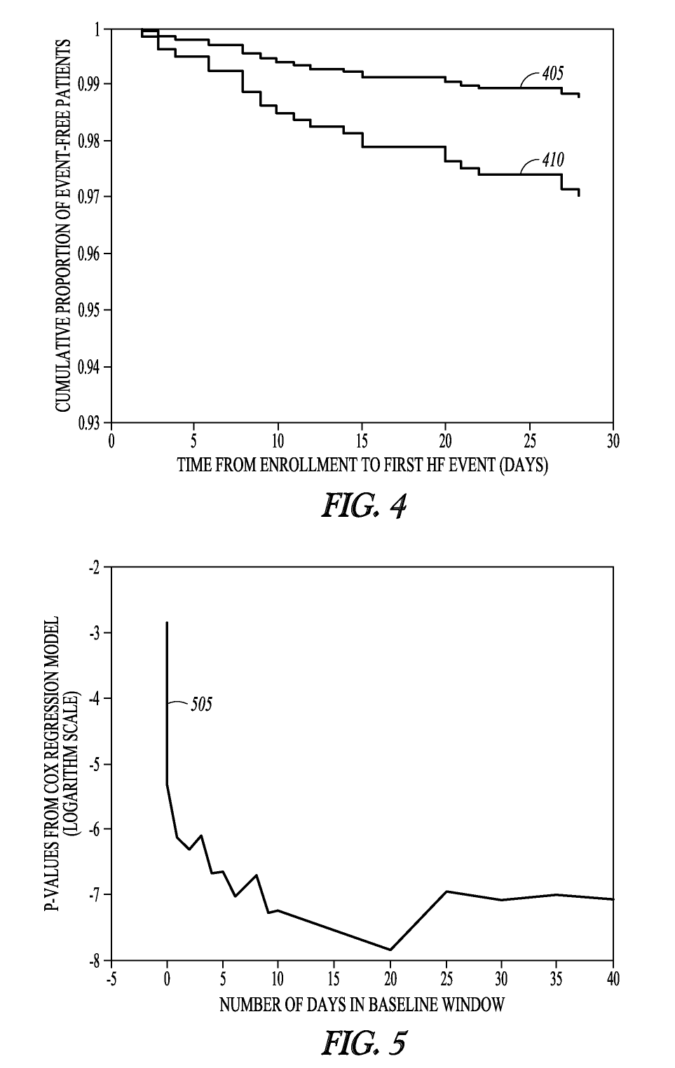

FIG. 4 shows an example of graphs of a proportion of a patient population that did not experience an acute HF event beginning with their time of first enrollment as an HF patient. The patients were divided into those with a high measurement of amplitude of the S3 heart sound and those with a low measurement of amplitude of the S3 heart sound. The graphs show that a greater proportion of patients with low S3 amplitude (graph 405) are event-free than patients with high S3 amplitude (graph 410). Thus, the graphs show that S3 amplitude can be used to assess risk of WHF.

FIG. 5 shows an example of a graph 505 of p-values from a regression model of the S3 energy data of the patient population. The horizontal axis represents the number of days of S3 energy data that was used to assess the risk of WHF to a patient. In the graph, S3 energy measurements averaged over more than one day resulted in lower p-values than when S3 energy measurements were averaged for less than one day of data. Lower p-values correspond to better separation of the risk data. Thus, averaging data over multiple days provides a better assessment of the risk of WHF. In the example of FIG. 5, the graph 505 shows that the p-values stabilize when data from 5 or more days were used.

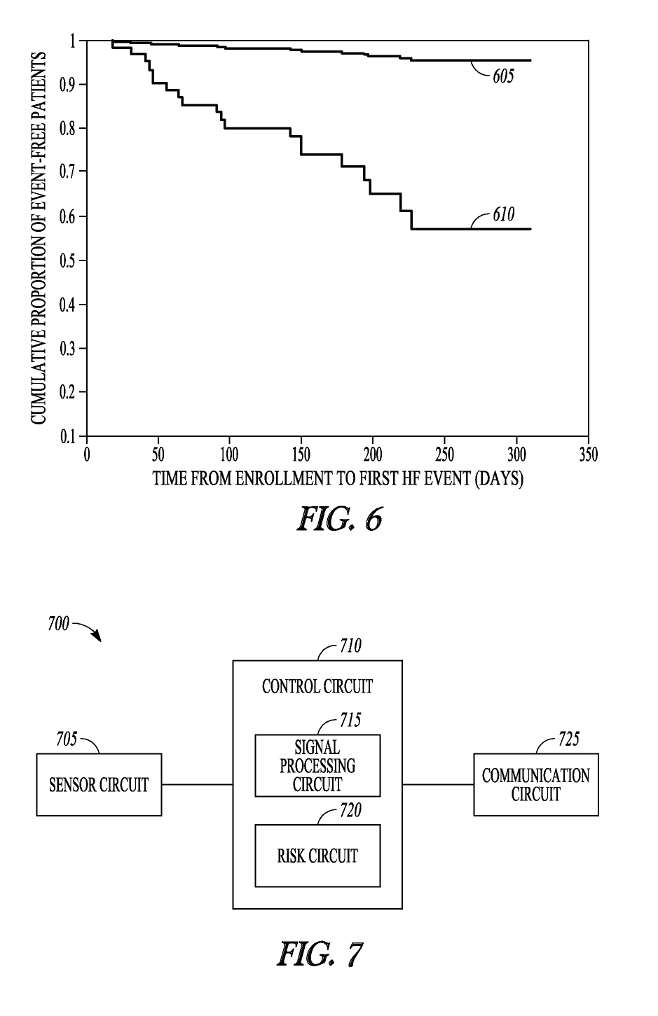

The quantified risk determined by the method of FIG. 3 is a reflection of the risk of the subject experiencing a heart failure event over the longer term (e.g., one to twelve months) rather than a risk of an acute HF event occurring during the next few minutes, the next hour, or later that same day. FIG. 6 shows an example of using a risk index for a patient population based on energy of the S3 heart sound. The Figure shows the proportion of the patient population that did not experience an acute HF event beginning with their time of first enrollment as an HF patient. The patients were divided into those with a high measurement of S3 heart sound energy and those with a low measurement of S3 heart sound energy. The graphs show a strong separation between the proportion of the low and high S3 energy groups that experienced an acute HF event between time of enrollment as an HF patient and more than 6 months after enrollment.

Assessing risk in the longer term can allow better allocation of resources for monitoring and treating HF while maintaining a high standard of care for all HF patients. For instance, if the central tendency measurement for the patient satisfies a risk criterion, the patient may be categorized as high risk and more monitoring resources maybe allocated to that patient. If the central tendency measurement for a patient does not satisfy a risk criterion, the patient may be categorized as low risk and resources allocated accordingly.

At block 330, an indication can be generated when the determined central tendency measurement satisfies the criteria indicative of risk of WHF. The indication can include an alert that presents a risk category for the subject on a display to the physician or caregiver. The indication can be provided to a process executing on a programming device or server. A follow-up schedule for the subject can be automatically adjusted according to the indication (e.g., follow-up visits can be made more frequent) or a suggested follow-up schedule can be presented for selection by a physician or caregiver.

FIG. 7 shows a block diagram of portions of an example of an ambulatory medical device 700 that assesses the risk of WHF for a subject. The device 700 includes at least a first physiological sensor circuit 705 and a control circuit 710 communicatively coupled to the physiological sensor circuit 705. The communicative coupling provides for electrical signals to be communicated between the physiological sensor circuit 705 and the communication circuit 710 even though there may be intervening circuitry between the physiological sensor circuit 705 and the control circuit 710.

The physiological sensor circuit 705 can generate a first physiological signal that is representative of cardiovascular function of a subject and a control circuit 710. An example of the physiological sensor circuit is a heart sound sensor circuit described previously herein. Another example of the physiologic sensor circuit 705 is a respiration sensor circuit. A respiration sensor circuit can generate a respiration signal that includes respiration information about the subject. The respiration signal can include any signal indicative of the respiration of the subject, such as inspiratory volume or flow, expiratory volume or flow, respiratory rate or timing, or any combination, permutation, or component of the respiration of the subject. A respiration sensor circuit can include an implantable sensor such as one or more of an accelerometer, an impedance sensor, a volume or flow sensor, and a pressure sensor.

Still another example of the physiological sensor circuit 705 is a cardiac signal sensor circuit. A cardiac signal sensor circuit generates a cardiac activity signal that is representative of electrical cardiac activity of the subject. An example of a cardiac signal sensor circuit includes one or more sense amplifiers connectable to one or more electrodes. Still another example of the physiological sensor circuit 705 is a biomarker sensor circuit. As explained previously herein, a biomarker sensor circuit generates a biomarker signal that is representative of a level of biomarker in the subject.

The control circuit 710 can include a microprocessor, a digital signal processor, application specific integrated circuit (ASIC), or other type of processor, interpreting or executing instructions in software modules or firmware modules. The control circuit 710 can include other circuits or sub-circuits to perform the functions described. These circuits may include software, hardware, firmware or any combination thereof. Multiple functions can be performed in one or more of the circuits and sub-circuits as desired.

The control circuit 710 includes a signal processing circuit 715 that is configured (e.g., by programming and/or by logic circuits) to determine a first physiological measurement using the first physiological sensor signal. As explained previously herein, if the physiological sensor circuit 705 includes a heart sound sensor circuit, the first physiological measurement can include a measurement of post-S2 heart sound energy. The measurement can include one or more of the amplitude, magnitude, and power of the post-S2 heart sound energy. In certain examples, the measurement includes a measurement of one or more of S3 heart sound energy and S4 heart sound energy.

The signal processing circuit 715 can determine a plurality of physiological measurements using a plurality of the physiological signals produced by the physiological sensor circuit 705 over a first specified time period (e.g., a number of days). The signal processing circuit 715 then determines a central tendency of the physiological measurement using the plurality of the physiological measurements.

The control circuit 710 can also include a risk circuit 720 that quantifies a risk of WHF for the subject using the determined central tendency measurement. In some examples, quantifying the risk of WHF includes comparing the determined central tendency measurement to one or more criteria indicative of risk of WHF. In some examples, the criteria include a comparison to one or more threshold values to determine a risk category of the subject. For instance, the risk circuit 720 may compare a central tendency measurement of S3 heart sound energy to a first S3 heart sound energy threshold value. If the central tendency measurement does not satisfy the first S3 heart sound energy threshold value, the subject may be placed into a low risk category. If the central tendency measurement satisfies the first S3 heart sound energy threshold value, the subject may be placed into a higher risk category.

More categories can be used in quantifying the risk. For instance, first and second S3 heart sound energy thresholds can be used with the second threshold value higher than the first. If the S3 central tendency measurement does not satisfy either the first S3 heart sound energy threshold value or the second S3 heart sound threshold energy value, the subject may be placed into a low risk category. If the S3 central tendency measurement satisfies the first S3 heart sound energy threshold value but not the second S3 heart sound energy threshold value, the subject may be placed into a medium risk category, and if the S3 central tendency measurement satisfies the second S3 heart sound energy threshold value, the subject may be placed into a high risk category. By extension, more categories can be used and the subject placed into a risk category according to the determined central tendency measurement.

In some examples, the risk circuit 720 quantifies the WHF risk by generating a risk index for the subject. The risk index may include the classifying of the risk of WHF of the subject as low, medium, or high risk. The risk index may include classifying the risk according to a risk quartile, decile, quintile, or the like. The risk index can be a continuous value (e.g., calculating a risk index for the subject as a probability with a value on a continuous scale of 0.0 to 1.0) indicating a degree of risk of an acute HF event. The risk index can be a raw measurement of physiologic sensor signal (such as, among other things, the raw measurement of amplitude of the S3 heart sound, the raw measurement of respiration rate variation, the raw measurement of the level of biomarker present in the subject, and the raw measurement of a time interval between features detected in one or more physiological signals).

As explained previously herein, the risk circuit 720 may compare the determined central tendency measurement to a first threshold risk detection value. The risk index can be a count of the number of times (e.g., the frequency) that the determined central tendency measurement satisfies the first threshold risk detection value within a specified period of time. The risk circuit 720 may determine the risk index recurrently, such as according to a schedule (such as daily, weekly, monthly, or even hourly). A notification may be generated according to the risk index.

A criterion indicative of risk of WHF (e.g., a threshold central tendency measurement value) used to generate the risk index can be specified (e.g., as a programmed value or a communicated value) to quantify the risk of an acute HF event occurring over a specified period, such as six months or twelve months for example. A risk criterion may be fixed once it is specified in the device 700, or the risk circuit 720 may recurrently execute an algorithm to adjust the one or more criteria for indicative of risk of WHF. For instance, the risk circuit 720 may adjust a risk criterion based on patient specific data (e.g., one or both of physiologic data and historical event data). In some examples, the threshold values can be programmable by a user (e.g., programmed according to preferences of a physician or according to data that is specific to the subject).

The control circuit 710 can generate an indication of the risk quantified by the risk circuit 720. For instance, the control circuit 710 may generate an indication of high risk based on a determined risk index. If the device 700 is included in a wearable device, the indication may be used to present an alert of the risk to user, such as by displaying the alert.

The device 700 may include a communication circuit 725 that communicates signals with a separate device. The communication may be via a wireless (e.g., RF telemetry) or wired (e.g., a universal serial bus) interface. The indication of risk may be communicated to a process on the separate device where an alert of high risk can be displayed or otherwise communicated, or a level of risk can be communicated to the process. In some examples, the separate device (e.g., a server) may adjust a schedule for follow-up visits of the subject based on the indication of risk. In some examples, the risk quantification is done by the separate device. For example, the risk circuit 720 may be included on the separate device and the device 700 communicates the measurements to the separate device where the risk is quantified.

In some examples, some preliminary signal processing can be performed on a physiological sensor signal before the signal is used in a determination of the central tendency measurement. For instance, the first physiological sensor circuit 705 may generate a first physiological sensor signal type. The signal processing circuit 715 may determine a central tendency signal (e.g., an ensemble average) using a plurality of signals of the first physiological sensor signal type obtained for a number of cardiac cycles. The signal processing circuit 715 determines a physiological measurement using a plurality of the central tendency signal (e.g., a measure of post-S2 heart sound energy is obtained from an ensemble average of heart sound signals) and a central tendency measurement is obtained using a plurality physiological measurements. As explained above, a central tendency signal is determined over a short time period, such as 30 seconds or using signals obtained from 8 to 10 cardiac cycles. The central tendency measurement is calculated using measurements taken over a time period of a day or more. The risk quantification is used to assess the risk of the subject experiencing WHF in the next few months to about a year.

Some examples of the central tendency measurement include a central tendency measurement of post-S2 heart sound energy, a central tendency measurement of S3 heart sound energy, a central tendency measurement of respiration rate, a central tendency measurement of the variation in respiration rate, a central tendency measurement of a level of biomarker detected in the subject, a central tendency measurement of a time interval between fiducial features in one or more physiological sensor signals, and a ratio of central tendency measurements of time intervals. Combinations of measurements can also be useful to assess risk of WHF.

According to some examples, the assessment of risk to an HF event can be made using both a central tendency measurement of post-S2 heart sound energy and a central tendency measurement of respiration rate. The first physiological sensor circuit 705 includes a heart sound sensor circuit and the device 700 includes a second physiological sensor circuit that includes a respiration sensor circuit. The signal processing circuit 715 determines a plurality of measurements of post-S2 heart sound energy using a plurality of heart sound signals and determines a plurality of measurements of respiration rate using a plurality of respiration signals. The signal processing circuit then determines a central tendency measurement of past-S2 heart sound energy and a central tendency measurement of respiration rate. The risk circuit quantifies the risk of WHF for the subject using the central tendency measurement of respiration rate and the central tendency measurement of post-S2 heart sound energy. In certain examples, the central tendency measurement of post-S2 heart sound energy can include a central tendency measurement of S3 energy and the central tendency measurement of respiration rate can include a central tendency of a measurement of variation in respiration rate.

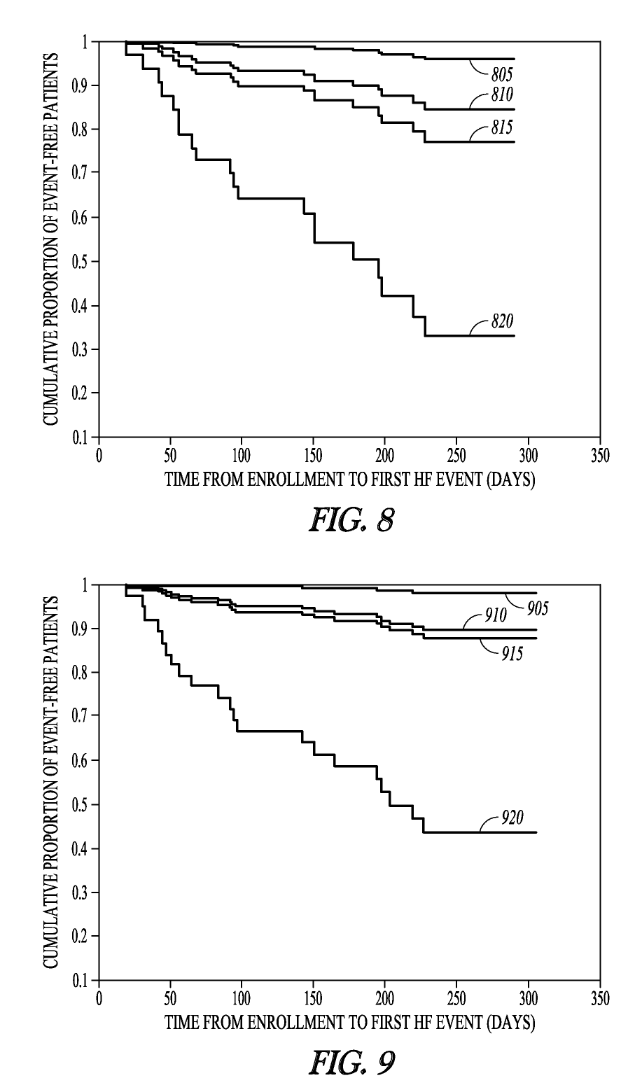

FIG. 8 shows an example of a risk index based on S3 energy and respiratory rate (RR) variation. The Figure shows graphs of the proportion of event-free patients for those patients with measured low S3 energy and measured low RR variation 805, low S3 energy and high RR variation 810, high S3 energy and low RR variation 815, and high S3 energy and high RR variation 820. The patients with measured low S3 energy and measured low RR variation may be placed in a low risk group and patients with measured high S3 energy and measured high RR variation may be placed in a high risk group. The remaining patients may be placed in a medium risk group. Determination of whether a central tendency measurement is low or high can include a comparison of the measurement to measurement threshold value. Indications of the risk of WHF can be used in one or more of displaying the risk assessment and changing the follow-up schedule of the patient. With low, medium, and high risk groups, three different levels of responses can be generated.

Other groupings for determining risk can be used (e.g., four individual risk groups) in assessing the risk of an HF event. Other methods of blending the sensors can also be used. For example, S3 energy may be given a different weight than RR variation in determining the risk index.

Other measurements from a heart sound signal can be used to quantify risk of WHF. For instance, a time interval measured between two fiducial features of the heart sound signal can be used in combination with one or more of the central tendency measurements of post-S2 heart sound energy and respiration rate. In some examples, the signal processing circuit 715 determines a time interval between two fiducial features of the heart sound signal and a plurality of the time intervals using a plurality of heart sound signals. The signal processing circuit 705 determines the central tendency measurement of the time intervals and the risk circuit quantifies the risk of WHF for the subject using the central tendency measurement of the time intervals and using at least one of the central tendency measurement of respiration rate and the central tendency measurement of post-S2 heart sound energy.

In some examples, the time interval is measured between a first fiducial feature indicating an S1 heart sound and a second fiducial feature indicating an S2 heart sound. The risk circuit 720 quantifies the risk of WHF for the subject using the central tendency measurement of a plurality of measured the time intervals between an S1 heart sound and an S2 heart sound and using at least one of the central tendency measurement of respiration rate and the central tendency measurement of post-S2 heart sound energy.

Other groupings of sensor data can be used. For instance, a time interval measured between two fiducial features of a sensed cardiac activity signal can be used in combination with one or more of the central tendency measurements of post-S2 heart sound energy and respiration rate. The first physiological sensor circuit 705 can include at least one of a heart sound sensor circuit or a respiration sensor circuit. The device 700 can include a second physiological sensor circuit that includes a cardiac signal sensor circuit. The signal processing circuit 715 measures a time interval between two fiducial features in the cardiac activity signal and determine a plurality of measurements of the time intervals using a plurality of cardiac activity signals. The signal processing circuit 715 determines a central tendency time interval using a plurality of measurements of the time interval. The signal processing circuit 715 also generates at least one of a central tendency post-S2 heart sound energy measurement or a central tendency respiration rate measurement. The risk circuit 720 quantifies a risk of WHF for the subject using the central tendency time interval and at least one of the central tendency post-S2 heart sound energy measurement or the central tendency respiration rate measurement.

In some examples, the fiducial features in the cardiac activity signal are R-waves, and the time interval in the cardiac activity signal includes a time interval from a first R-wave to a second R-wave. The risk circuit 720 quantifies a risk of WHF for the subject using the central tendency of measured R-wave to R-wave time intervals and at least one of the central tendency post-S2 heart sound energy measurement or the central tendency respiration rate measurement.

In another sensor data grouping, a time interval measured between at least one fiducial feature of a sensed cardiac activity signal and at least one fiducial feature in a sensed heart sound signal can be used in combination with one or more of the central tendency measurements of post-S2 heart sound energy and respiration rate. The first physiological sensor circuit 705 can include a heart sound sensor circuit, and the device 700 includes a second physiological sensor circuit that includes a respiration sensor circuit and a third physiological sensor circuit that includes a cardiac signal sensor circuit.

The signal processing circuit 715 measures a time interval between a fiducial feature in a cardiac activity signal and a fiducial feature in a heart sound signal and determine a plurality of measurements of the time intervals using a plurality of cardiac activity signals and heart sound signals. The signal processing circuit 705 measures a central tendency time interval using the plurality of time interval measurements, and determines at least one of a central tendency measurement of post-S2 heart sound energy using a plurality of post-S2 heart sound energy obtained from a plurality of heart sound signals or a central tendency measurement of respiration rate using a plurality of respiration rate measurements obtained from a plurality of respiration signals. The risk circuit 720 quantifies a risk of WHF for the subject using the central tendency time interval and at least one of the central tendency post-S2 heart sound energy measurement or the central tendency respiration rate measurement.

The time interval between the fiducial feature in the cardiac activity signal and the fiducial feature in the heart sound signal can include at least one of i) a time interval between an R-wave and an S1 heart sound, ii) a time interval between an Q-wave and an S1 heart sound, iii) a time interval between a R-wave and a fiducial representative of opening of the aortic valve (Ao), iv) a time interval between a Q-wave and a fiducial representative of Ao, or v) a time interval between a fiducial feature representative of Ao and a fiducial feature representative of closing of the aortic valve (Ac).

Ratios of time intervals can be used. The signal processing circuit 715 may determine the central tendency of two of the time intervals and determine of a ratio of the central tendency measurements.

In another sensor data grouping, a measure of the level of a biomarker present in the subject can be used in combination with at least one of a measure of post-S2 heart sound energy, a measure of respiration rate, or a measure of a time interval to assess risk of WHF. The first physiological sensor circuit 705 includes at least one of a heart sound sensor circuit, a respiration sensor circuit, or a cardiac signal sensor circuit. The device 700 includes second physiological sensor circuit that includes a biomarker sensor circuit.

The signal processing circuit 715 determines a plurality of indications of the level of biomarker in the subject using a plurality of biomarker signals and generates a central tendency of the indication of the biomarker level using the plurality of indications of the level of biomarker. The signal processing circuit 715 also generates at least one of a central tendency post-S2 heart sound energy measurement, a central tendency respiration rate measurement, a central tendency measurement of a time interval between two fiducial features in a heart sound signal, a central tendency measurement of a time interval between two fiducial features in a cardiac activity signal, or a central tendency measurement of a time interval between a fiducial feature in a cardiac signal and a fiducial feature in a heart sound signal.

The risk circuit 720 quantifies the risk of WHF for the subject using central tendency of the indication of the biomarker level and at least one of the central tendency post-S2 heart sound energy measurement, the central tendency respiration rate measurement, the central tendency measurement of a time interval between two fiducial features in a heart sound signal, the central tendency measurement of a time interval between two fiducial features in a cardiac activity signal, or the central tendency measurement of a time interval between a fiducial feature in a cardiac signal and a fiducial feature in a heart sound signal.

According to some examples, historical HF data can be used in assessing the risk of an HF event. The risk circuit 720 quantifies the risk of WHF for the subject using a determined central tendency measurement (e.g., a central tendency measurement of post-S2 heart sound energy) and using historical data of HF admissions for the subject. In some examples, the criteria indicative of risk of WHF can include a first threshold risk detection value for the determined central tendency measurement. The risk circuit 720 can adjust the first threshold risk detection value according to one or both of physiologic data and historical data of HF admissions for the subject. The historical data may be stored in a memory integral to or coupled to the control circuit 710, of the historical data may be stored in a separate device.

FIG. 9 shows an example of a risk index determined using S3 energy and history of HF admissions. An HF admission refers to whether the patient received treatment for HF in a hospital or as an outpatient. In some examples, the HF admission may be positive or true if the patient received at least one treatment in the last six months or received at least two treatments in the last twelve months. The Figure shows graphs of the proportion of event-free patients for those patients with a measure of low S3 energy and no HF admission in their history 905, a measure of low S3 energy and an HF admission in their history 910, a measure of high S3 energy and no HF admission in their history 915, and a measure of high S3 energy and an HF admission in their history 920. The patients with low S3 energy and no HF admission history can be placed in a low risk group and patients with high S3 energy and with HF admission history can be placed in a high risk group. The remaining patients can be placed in a medium risk group to create three levels of responses generated, or the other patients may be placed in the low risk group. If the subject history includes several episodes of HF admissions, the risk circuit 720 may adjust one or more threshold risk detection values to increase the sensitivity of the assessment. Similarly, if the subject history includes a low number or no episodes of HF admissions, the risk circuit 720 may adjust one or more threshold risk detection values to lower the sensitivity of the assessment.

Other examples include assessing risk using HF admission history and at least one of a central tendency measurement of respiration rate and HF admission history, a central tendency measurement of a biomarker level and HF admission history, a central tendency measurement of a time interval between fiducial features of one or more physiological signals, or using any combination of post-S2 heart sound energy, respiration rate, biomarker level, and time intervals.

These several examples of devices and methods show that monitoring physiologic events of a subject can be useful in predicting the risk that the subject will experience worsening heart failure in the future. This allows for efficient allocation of health care resources to monitor and treat HF in patients.

### ADDITIONAL NOTES AND EXAMPLES

Example 1 can include or use subject matter (such as an apparatus, a device, or a system) comprising at least a first physiological sensor circuit configured to generate a first physiological signal that is representative of cardiovascular function of a subject and a control circuit communicatively coupled to the first physiological sensor circuit. The control circuit includes a signal processing circuit and a risk circuit. The signal processing circuit is configured to determine a first physiological measurement using the first physiological sensor signal and determine a plurality of the first physiological measurements using a plurality of first physiological signals produced over a first specified time period, and determine a central tendency measurement of the plurality of physiological measurements. The risk circuit is configured to quantify a risk of worsening heart failure (WHF) for the subject using the determined central tendency measurement, including comparing the determined central tendency measurement to one or more criteria indicative of risk of WHF. The control circuit is configured to generate an alert when the central tendency measurement satisfies the one or more criteria indicative of risk of WHF.

Example 2 can include, or can optionally be combined with the subject matter of Example 1 to include a first physiological sensor circuit configured to generate a first physiological signal type, and a signal processing circuit optionally configured to generate a first central tendency signal using a plurality of signals of the first physiological sensor signal type obtained for a number of cardiac cycles, and determine the first physiological measurement using the first central tendency signal.

Example 3 can include, or can optionally be combined with the subject matter of one or any combination of Examples 1 and 2 to include a first specified time period that includes a number of days.

Example 4 can include, or can optionally be combined with the subject matter of one or any combination of Examples 1 through 3 to include a physiological sensor circuit that includes a heart sound sensor circuit configured to generate a heart sound signal that is representative of mechanical activity of a heart of the subject. The signal processing circuit can optionally be configured to determine a measurement of post-S2 heart sound energy using the heart sound signal and a plurality of measurements of post-S2 heart sound energy using a plurality of heart sound signals, and determine a central tendency measurement of post-S2 heart sound energy. The risk circuit can optionally be configured to quantify the risk of WHF for the subject using the central tendency measurement of post-S2 heart sound energy.

Example 5 can include or can optionally be combined with the subject matter of Example 4 to include a physiological sensor circuit that includes a respiration sensor circuit configured to generate a respiration signal that is representative of respiration of the subject. The signal processing circuit can optionally be configured to determine a measurement of respiration rate using the respiration signal and a plurality of measurements of respiration rate using a plurality of respiration signals, and determine a central tendency measurement of respiration rate. The risk circuit can optionally be configured to quantify the risk of WHF for the subject using the central tendency measurement of respiration rate and the central tendency measurement of post-S2 heart sound energy.

Example 6 can include, or can optionally be combined with the subject matter of Example 5 to include a signal processing circuit configured to determine a variation in respiration rate using the plurality of measurements of respiration rate, and a risk circuit configured to quantify the risk of WHF for the subject using the variation of the respiration rate and the central tendency measurement of post-S2 heart sound energy.

Example 7 can include, or can optionally be combined with the subject matter of one or any combination of Examples 4 through 6 to include a signal processing circuit is configured to determine a measurement of S3 heart sound energy using the heart sound signal and a plurality of measurements of S3 heart sound energy using a plurality of heart sound signals, and determine a central tendency measurement of S3 heart sound energy. The risk circuit can optionally be configured to quantify a risk of WHF for the subject using the central tendency measurement of S3 heart sound energy.

Example 8 can include, or can optionally be combined with the subject matter of one or any combination of Examples 1 through 3 to include a first physiological sensor circuit includes a heart sound sensor circuit configured to generate a heart sound signal that is representative of mechanical activity of a heart of the subject, a second physiological sensor circuit that includes a respiration sensor circuit configured to generate a respiration signal that is representative of respiration of the subject, and a third physiological sensor circuit that includes a cardiac signal sensor circuit configured to generate a cardiac activity signal representative of electrical cardiac activity of the subject. The signal processing circuit can optionally be configured to determine at least one of a plurality of measurements of post-S2 heart sound energy using a plurality of heart sound signals or a plurality of measurements of respiration rate using a plurality of respiration signals, generate at least one of a central tendency post-S2 heart sound energy measurement or a central tendency respiration rate measurement, measure one or more time intervals between at least one fiducial feature in a cardiac activity signal and at least one fiducial feature in a heart sound signal and determine a plurality of measurements of the time intervals using a plurality of cardiac activity signals and heart sound signals, and determine, using the plurality of measurements of the time intervals, at least one of a central tendency time interval or a central tendency of a ratio of time intervals. The risk circuit can optionally be configured to quantify a risk of WHF for the subject using the central tendency time interval and at least one of the central tendency post-S2 heart sound energy measurement or the central tendency respiration rate measurement.

Example 9 can include, or can optionally be combined with the subject matter of Example 8 to include measured time intervals between the at least one fiducial feature in the cardiac activity signal and the at least one fiducial feature in the heart sound signal that include at least one of a time interval between an R-wave and an S1 heart sound, a time interval between an Q-wave and an S1 heart sound, a time interval between an R-wave and R-wave, a time interval between an Q-wave and Q-wave, a time interval between an S1 heart sound and an S2 heart sound, a time interval between an R-wave and an S2 heart sound, a time interval between an Q-wave and an S2 heart sound, a time interval between a R-wave and a fiducial representative of opening of the aortic valve (Ao), a time interval between a Q-wave and a fiducial representative of Ao, or a time interval between a fiducial feature representative of Ao and a fiducial feature representative of closing of the aortic valve (Ac).

Example 10 can include, or can optionally be combined with the subject matter of one or any combination of Examples 1-3 to include a first physiological sensor circuit includes at least one of a heart sound sensor circuit configured to generate a heart sound signal that is representative of mechanical activation of a chamber of a heart of the subject, a respiration sensor circuit configured to generate a respiration signal that is representative of respiration of the subject, or a cardiac signal sensor circuit configured to generate a cardiac signal representative of electrical cardiac activity of the subject, and a second physiological sensor circuit that includes a biomarker sensor circuit configured to generate a biomarker signal that is representative of a level of biomarker in the subject. The signal processing circuit can optionally be configured to determine at least one of a plurality of measurements of post-S2 heart sound energy using a plurality of heart sound signals, a plurality of measurements of respiration rate using a plurality of respiration signals, a plurality of measurements of a time interval between two fiducial features in a heart sound signal, a plurality of measurements of a time interval between two fiducial features in a cardiac activity signal, or a plurality of measurements of a time interval between a fiducial feature in a cardiac signal and a fiducial feature in a heart sound signal. The signal processing circuit can optionally be configured to generate at least one of a central tendency post-S2 heart sound energy measurement, a central tendency respiration rate measurement, a central tendency measurement of a time interval between two fiducial features in a heart sound signal, a central tendency measurement of a time interval between two fiducial features in a cardiac activity signal, or a central tendency measurement of a time interval between a fiducial feature in a cardiac signal and a fiducial feature in a heart sound signal. The signal processing circuit can optionally be configured to determine a plurality of indications of the level of biomarker in the subject using a plurality of biomarker signals, and generate a central tendency of the indication of the biomarker level using the plurality of indications of the level of biomarker. The risk circuit can optionally be configured to quantify the risk of WHF for the subject using central tendency of the indication of the biomarker level and at least one of the central tendency post-S2 heart sound energy measurement, the central tendency respiration rate measurement, the central tendency measurement of a time interval between two fiducial features in a heart sound signal, the central tendency measurement of a time interval between two fiducial features in a cardiac activity signal, or the central tendency measurement of a time interval between a fiducial feature in a cardiac signal and a fiducial feature in a heart sound signal.

Example 11 can include, or can optionally be combined with the subject matter of Example 10 to include a biomarker sensor circuit configured to generate a biomarker signal that is representative of at least one of a level of B-type Naturetic Peptide (BNP) in the subject, or a level of NT-Pro-BNP of the subject.

Example 12 can include, or can optionally be combined with the subject matter of one or any combination of Examples 1-11 to include a risk circuit configured to quantify the risk of WHF for the subject using the determined central tendency measurement and using historical data of HF admissions for the subject.

Example 13 can include, or can optionally be combined with the subject matter of one or any combination of Examples 1-12 to include a risk circuit configured to compare the determined central tendency measurement to a first threshold risk detection value, and determine a risk index for WHF according to a frequency with which the determined central tendency measurement satisfies the first threshold risk detection value within a specified period of time, wherein the control circuit is configured to generate the alert according to the risk index.

Example 14 can include, or can optionally be combined with the subject matter of one or any combination of Examples 1-13 to include criteria indicative of risk of WHF that includes a first threshold risk detection value for the determined central tendency measurement, and a risk circuit optionally configured to adjust the first threshold risk detection value according to one or both of physiologic data and historical data of HF admissions for the subject.

Example 15 can include, or can optionally be combined with the subject matter of one or any combination of Examples 1-14 to include a risk circuit configured to recurrently quantify a risk of WHF for the subject and recurrently adjust the one or more criteria for indicative of risk of WHF.

Example 16 can include, or can optionally be combined with the subject matter of one or any combination of Examples 1-15 to include subject matter (such as a method of operating a device, a means for performing acts, or a machine readable medium including instructions that, when performed by the machine, cause the machine to perform acts) comprising producing a first physiological sensor signal that is representative of cardiovascular function using a first physiological sensor of an ambulatory medical device, determining a first physiological measurement using the first physiological sensor signal, producing a plurality of the first physiological sensor signals over a first specified time period and determining a plurality of physiological measurements using the plurality of first physiological sensor signals, determining a central tendency measurement of the plurality of physiological measurements, and quantifying a risk of WHF for the subject using the determined central tendency measurement. Quantifying the risk of WHF can optionally include comparing the determined central tendency measurement to one or more criteria indicative of risk of WHF. The subject matter can optionally include generating an alert by the device when the determined central tendency measurement satisfies the criteria indicative of risk of WHF.

Example 17 can include, or can optionally be combined with the subject matter of Example 16 to include producing a plurality of heart sound signals, determining a plurality of measurements of post-S2 heart sound energy using the plurality of heart sound signals, determining a central tendency measurement of post-S2 heart sound energy, and quantifying a risk of WHF for the subject using the central tendency measurement of post-S2 heart sound energy.

Example 18 can include, or can optionally be combined with the subject matter of one or any combination of Examples 16 and 17 to include producing a plurality of respiration signals using a respiration sensor circuit, determining a plurality of measurements of respiration rate using the plurality of respiration signals, determining a central tendency measurement of respiration rate using the plurality of measurements of respiration rate, and quantifying the risk of WHF for the subject using the central tendency measurement of post-S2 heart sound energy and the central tendency measurement of respiration rate.

Example 19 can include, or can optionally be combined with the subject matter of Example 16 to optionally include producing at least one of a plurality of heart sound signals or a plurality of respiration signals, wherein a heart sound signal is representative of mechanical activity of a heart of the subject and a respiration signal is representative of respiration of a subject, determining at least one of a plurality of measurements of post-S2 heart sound energy or a plurality of measurements of respiration rate, determining a central tendency measurement includes determining at least one of a central tendency post-S2 heart sound energy measurement or a central tendency respiration rate measurement, producing a plurality of cardiac activity signals, wherein a cardiac activity signal is representative of electrical cardiac activity of the subject, determining a plurality of measurements for a time interval between at least one fiducial feature in a heart sound signal and at least one fiducial feature in a cardiac activity signal, and determining a central tendency measurement of the time interval between the at least one fiducial feature in a heart sound signal and the at least one fiducial feature in a cardiac activity signal. The subject matter optionally includes quantifying the risk of WHF for the subject using the central tendency measurement of the time interval and the at least one of the central tendency post-S2 heart sound energy measurement or the central tendency respiration rate measurement.

Example 20 can include, or can optionally be combined with the subject matter of one or any combination of Examples 16-19 to include storing historical data of HF admissions for the subject, and quantifying the risk of WHF for the subject using the determined central tendency measurement and the historical data of HF admissions for the subject.

Example 21 can include, or can optionally be combined with any portion or combination of any portions of any one or more of Examples 1 through 20 to include, subject matter that can include means for performing any one or more of the functions of Examples 1 through 20, or a machine-readable medium including instructions that, when performed by a machine, cause the machine to perform any one or more of the functions of Examples 1 through 20.

The above detailed description includes references to the accompanying drawings, which form a part of the detailed description. The drawings show, by way of illustration, specific embodiments in which the invention can be practiced. These embodiments are also referred to herein as “examples.” In the event of inconsistent usages between this document and any documents incorporated by reference, the usage in the incorporated reference(s) should be considered supplementary to that of this document; for irreconcilable inconsistencies, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In the appended claims, the terms “including” and “in which” are used as the plain-English equivalents of the respective terms “comprising” and “wherein.” Also, in the following claims, the terms “including” and “comprising” are open-ended, that is, a system, device, article, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code can form portions of computer program products. Further, the code can be tangibly stored on one or more volatile or non-volatile computer-readable media during execution or at other times. These computer-readable media can include, but are not limited to, hard disks, removable magnetic disks, removable optical disks (e.g., compact disks and digital video disks), magnetic cassettes, memory cards or sticks, random access memories (RAM's), read only memories (ROM's), and the like. In some examples, a carrier medium can carry code implementing the methods. The term “carrier medium” can be used to represent carrier waves on which code is transmitted.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to comply with 37 C.F.R. §1.72(b), to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description, with each claim standing on its own as a separate embodiment. The scope of the invention should be determined with reference to the appended claims, along with the full scope of equivalents to which such claims are entitled.

What is claimed is:

1. An apparatus comprising:
at least a first physiological sensor circuit configured to generate a first physiological signal that is representative of cardiovascular function of a subject;
a control circuit communicatively coupled to the first physiological sensor circuit, wherein the control circuit includes:
a signal processing circuit configured to:
determine a first physiological measurement using the first physiological sensor signal and determine a plurality of the first physiological measurements using a plurality of first physiological signals produced over a specified first time period; and
determine a central tendency measurement of the plurality of physiological measurements; and
a risk circuit configured to quantify a risk of worsening heart failure (WHF) for the subject using the determined central tendency measurement, including comparing the determined central tendency measurement to one or more criteria indicative of risk of WHF,
wherein the control circuit is configured to generate an indication of risk of WHF according to a comparison of the determined central tendency measurement to the one or more criteria indicative of risk of WHF.

2. The apparatus of claim 1,
wherein the first physiological sensor circuit is configured to generate a first physiological signal type, and
wherein the signal processing circuit is configured to:
generate a first central tendency signal using a plurality of signals of the first physiological sensor signal type obtained for a number of cardiac cycles; and
determine the first physiological measurement using the first central tendency signal.

3. The apparatus of claim 1, wherein the first time period includes a number of days.

4. The apparatus of claim 1,
wherein the first physiological sensor circuit includes a heart sound sensor circuit configured to generate a heart sound signal that is representative of mechanical activity of a heart of the subject,
wherein the signal processing circuit is configured to:
determine a measurement of post-S2 heart sound energy using the heart sound signal and a plurality of measurements of post-S2 heart sound energy using a plurality of heart sound signals; and
determine a central tendency measurement of post-S2 heart sound energy, and
wherein the risk circuit is configured to quantify the risk of WHF for the subject using the central tendency measurement of post-S2 heart sound energy.

5. The apparatus of claim 4, including:
a second physiological sensor circuit that includes a respiration sensor circuit configured to generate a respiration signal that is representative of respiration of the subject,
wherein the signal processing circuit is configured to:
determine a measurement of respiration rate using the respiration signal and a plurality of measurements of respiration rate using a plurality of respiration signals; and
determine a central tendency measurement of respiration rate; and
wherein the risk circuit is configured to quantify the risk of WHF for the subject using the central tendency measurement of respiration rate and the central tendency measurement of post-S2 heart sound energy.

6. The apparatus of claim 5,
wherein the signal processing circuit is configured to determine a variation in respiration rate using the plurality of measurements of respiration rate, and
wherein the risk circuit is configured to quantify the risk of WHF for the subject using the variation of the respiration rate and the central tendency measurement of post-S2 heart sound energy.

7. The apparatus of claim 4,
wherein the signal processing circuit is configured to:
determine a measurement of S3 heart sound energy using the heart sound signal and a plurality of measurements of S3 heart sound energy using a plurality of heart sound signals; and
determine a central tendency measurement of S3 heart sound energy, and
wherein the risk circuit is configured to quantify a risk of WHF for the subject using the central tendency measurement of S3 heart sound energy.

8. The apparatus of claim 1,
wherein the first physiological sensor circuit includes a heart sound sensor circuit configured to generate a heart sound signal that is representative of mechanical activity of a heart of the subject,
wherein the apparatus includes a second physiological sensor circuit that includes a respiration sensor circuit configured to generate a respiration signal that is representative of respiration of the subject, and a third physiological sensor circuit that includes a cardiac signal sensor circuit configured to generate a cardiac activity signal representative of electrical cardiac activity of the subject,
wherein the signal processing circuit is configured to:
determine at least one of a plurality of measurements of post-S2 heart sound energy using a plurality of heart sound signals or a plurality of measurements of respiration rate using a plurality of respiration signals;
generate at least one of a central tendency post-S2 heart sound energy measurement or a central tendency respiration rate measurement;
measure one or more time intervals between at least one fiducial feature in a cardiac activity signal and at least one fiducial feature in a heart sound signal and determine a plurality of measurements of the time intervals using a plurality of cardiac activity signals and heart sound signals; and
determine, using the plurality of measurements of the time intervals, at least one of a central tendency time interval or a central tendency of a ratio of time intervals,
wherein the risk circuit is configured to quantify a risk of WHF for the subject using the central tendency time interval and at least one of the central tendency post-S2 heart sound energy measurement or the central tendency respiration rate measurement.

9. The apparatus of claim 8, wherein the time intervals between the at least one fiducial feature in the cardiac activity signal and the at least one fiducial feature in the heart sound signal includes at least one of:
a time interval between an R-wave and an S1 heart sound;
a time interval between an Q-wave and an S1 heart sound;
a time interval between an R-wave and R-wave;
a time interval between an Q-wave and Q-wave;
a time interval between an S1 heart sound and an S2 heart sound;
a time interval between an R-wave and an S2 heart sound;
a time interval between an Q-wave and an S2 heart sound;
a time interval between a R-wave and a fiducial representative of opening of the aortic valve (Ao);
a time interval between a Q-wave and a fiducial representative of Ao; or
a time interval between a fiducial feature representative of Ao and a fiducial feature representative of closing of the aortic valve (Ac).

10. The apparatus of claim 1, wherein the first physiological sensor circuit includes at least one of:
a heart sound sensor circuit configured to generate a heart sound signal that is representative of mechanical activation of a chamber of a heart of the subject;
a respiration sensor circuit configured to generate a respiration signal that is representative of respiration of the subject; or
a cardiac signal sensor circuit configured to generate a cardiac signal representative of electrical cardiac activity of the subject.

11. The apparatus of claim 10,
wherein the apparatus includes a second physiological sensor circuit that includes a biomarker sensor circuit configured to generate a biomarker signal that is representative of a level of biomarker in the subject,
wherein the signal processing circuit is configured to:
determine at least one of a plurality of measurements of post-S2 heart sound energy using a plurality of heart sound signals, a plurality of measurements of respiration rate using a plurality of respiration signals, a plurality of measurements of a time interval between two fiducial features in a heart sound signal, a plurality of measurements of a time interval between two fiducial features in a cardiac activity signal, or a plurality of measurements of a time interval between a fiducial feature in a cardiac signal and a fiducial feature in a heart sound signal;
generate at least one of a central tendency post-S2 heart sound energy measurement, a central tendency respiration rate measurement, a central tendency measurement of a time interval between two fiducial features in a heart sound signal, a central tendency measurement of a time interval between two fiducial features in a cardiac activity signal, or a central tendency measurement of a time interval between a fiducial feature in a cardiac signal and a fiducial feature in a heart sound signal;
determine a plurality of indications of the level of biomarker in the subject using a plurality of biomarker signals; and
generate a central tendency of the indication of the biomarker level using the plurality of indications of the level of biomarker,
wherein the risk circuit is configured to quantify the risk of WHF for the subject using central tendency of the indication of the biomarker level and at least one of the central tendency post-S2 heart sound energy measurement, the central tendency respiration rate measurement, the central tendency measurement of a time interval between two fiducial features in a heart sound signal, the central tendency measurement of a time interval between two fiducial features in a cardiac activity signal, or the central tendency measurement of a time interval between a fiducial feature in a cardiac signal and a fiducial feature in a heart sound signal.

12. The apparatus of claim 11, wherein the biomarker sensor circuit is configured to generate a biomarker signal that is representative of at least one of:
a level of B-type Naturetic Peptide (BNP) in the subject; or
a level of NT-Pro-BNP of the subject.

13. The apparatus of claim 1, wherein the risk circuit is configured to quantify the risk of WHF for the subject using the determined central tendency measurement and using historical data of HF admissions for the subject.

14. The apparatus of claim 1, wherein the risk circuit is configured to:
compare the determined central tendency measurement to a first threshold risk detection value; and
determine a risk index for WHF according to a frequency with which the determined central tendency measurement satisfies the first threshold risk detection value within a specified period of time, wherein the control circuit is configured to generate the alert according to the risk index.

15. The apparatus of claim 1,
wherein the criteria indicative of risk of WHF includes a first threshold risk detection value for the determined central tendency measurement, and
wherein the risk circuit is configured to adjust the first threshold risk detection value according to one or both of physiologic data and historical data of HF admissions for the subject.

16. The apparatus of claim 1, wherein the risk circuit is configured to recurrently quantify a risk of WHF for the subject and recurrently adjust the one or more criteria indicative of risk of WHF.

17. A method of operating an ambulatory medical device, the method comprising:
producing a first physiological sensor signal using a first physiological sensor of the ambulatory medical device, wherein a physiological sensor signal is representative of cardiovascular function of a subject;
determining a first physiological measurement using the first physiological sensor signal;
producing a plurality of the first physiological sensor signals over a specified first time period and determining a plurality of physiological measurements using the plurality of first physiological sensor signals;
determining a central tendency measurement of the plurality of physiological measurements;
quantifying a risk of WHF for the subject using the determined central tendency measurement, including comparing the determined central tendency measurement to one or more criteria indicative of risk of WHF; and
generating an indication of risk of WHF according to a comparison of the determined central tendency measurement to the one or more criteria indicative of risk of WHF.

18. The method of claim 17,
wherein producing a plurality of the first physiological sensor signals includes producing a plurality of heart sound signals, wherein a heart sound signal is representative of mechanical activity of a heart of the subject,
wherein determining a plurality of physiological measurements includes determining a plurality of measurements of post-S2 heart sound energy using the plurality of heart sound signals,
wherein determining a central tendency measurement includes determining a central tendency measurement of post-S2 heart sound energy, and
wherein quantifying a risk of WHF includes quantifying a risk of WHF for the subject using the central tendency measurement of post-S2 heart sound energy.

19. The method of claim 18, including:
producing a plurality of respiration signals using a respiration sensor circuit, wherein a respiration signal is representative of respiration of the subject;
determining a plurality of measurements of respiration rate using the plurality of respiration signals;
determining a central tendency measurement of respiration rate using the plurality of measurements of respiration rate, and
wherein quantifying the risk of WHF includes quantifying the risk of WHF for the subject using the central tendency measurement of post-S2 heart sound energy and the central tendency measurement of respiration rate.

20. The method of claim 19, including storing historical data of HF admissions for the subject, and wherein quantifying the risk of WHF includes quantifying the risk of WHF for the subject using the determined central tendency measurement and the historical data of HF admissions for the subject.
